(19) United States
(12) Patent Application Publication
(10) Pub. No.: US 2012/0082997 A1
(43) Pub. Date: Apr. 5, 2012

(54) TAGGANTS AND METHOD OF USING SAME

(75) Inventors: Ehud Keinan, Timrat (IL); Richard A. Lerner, La Jolla, CA (US)

(73) Assignee: Technion Research & Development, Haifa (IL)

(21) Appl. No.: 13/154,457

(22) Filed: Jun. 7, 2011

Related U.S. Application Data
(60) Provisional application No. 61/352,430, filed on Jun. 8, 2010.

(51) Int. Cl. C12Q 1/68

(52) U.S. Cl. 435/6.15

(57) ABSTRACT

A composition of matter which comprises a non-gaseous nitrogen containing compound; and a spore of a micro-organism. A method of determining a source of a material the material being an explosive or a substance used in the preparation of the explosive, the material comprises a spore of a micro-organism uniquely associated with the material, the method comprises inducing the spore to transform into a vegetative micro-organism and identifying the micro-organism as being associated with the material, thereby determining the source of the material.

This application claims the benefit of priority under 35 USC 119(e) of U.S. Provisional Patent Application No. 61/352,430 filed Jun. 8, 2010, the contents of which are incorporated herein by reference in their entirety.

### FIELD AND BACKGROUND OF THE INVENTION

The present invention, in some embodiments thereof, relates to a taggant and its uses, and more particularly, but not exclusively, to (i) a taggant incorporated in explosives and in substances used in the preparation of explosives, (ii) explosives and substances used in the preparation of explosives incorporating the taggant; (iii) methods for their production; and (iv) methods of identifying the source of an explosive, before or after detonation or blast, and (v) the source of substances used in the preparation of explosives. The present invention, in some other embodiments thereof, relates to a taggant and its uses, and more particularly, but not exclusively, to (i) a taggant incorporated in narcotics and in substances used in the preparation of narcotics, (ii) narcotics and substances used in the preparation of narcotics incorporating the taggant; (iii) methods for their production; and (iv) methods of identifying the source of a narcotic; and (v) the source of substances used in the preparation of narcotics.

Detectable taggant material, usually in the form of volatile chemical markers added to commercially made explosives, were introduced as a result of international treaties. For example, according to the international treaty administered by the International Civil Aviation Organization (ICAO) in reaction to the bombing of Pan Am Flight 103 over Lockerbie, Scotland in 1988, “detection markers” are used to help improve the detection of explosives prior to detonation. There is a choice between four possible chemical taggants. In the United States the taggant is 2,3-dimethyl-2,3-dinitrobutane (DMNB). Dogs are very sensitive to it and can detect as little as 0.5 ppb in air, as can specialized ion mobility spectrometers. Other taggants in use are ethylene glycol dinitrate (EGDN) used to mark Semtex, ortho-mononitrotoluene (o-MNT), and para-mononitrotoluene (p-MNT).

In a joint statement of Nov. 30, 2007, Cefic (Conseil Européen de l'Industrie Chimique/European Chemical Industry Council) and FECC (European Association of Chemical Distributors) pointed out the following: “ . . . . Only small amounts may be required (less than one tenth of a liter or a kilogram) to make a detonator but the production and trade volumes of these chemicals are tremendous; millions of tons are produced/used and sold all over the world. It is a global business. Industry and genuine end-users could not possibly introduce stock control for the loss/theft/usage of such small amounts.”

Ammonium nitrate (AN) is mainly used as a fertilizer, but also as the main ingredient in civil explosives. For use in civil explosives there are no real alternatives, but for this application the logistic chain is quite good with deliveries direct from ammonium nitrate manufacturer to explosives companies. For fertilizers the chain is weaker with the end user, the farmer, being the weakest link. Also transport from distributor to small farms can be questionable. On the other hand there is no real good alternative to ammonium nitrate as fertilizer, especially in cold climates.

Urea is used in the preparation of urea nitrate (UN), which is another improvised explosive, easily prepared from a common fertilizer and nitric acid.

Adding taggants to explosives and substances used for their preparation, such as ammonium nitrate and urea nitrate, would increase the probability of tracking of their manufacturer and origin in case of terror attacks and other illicit uses. This approach also opens the door for voluntary commitments from the industry to take measures to improve the security of the products most easily available, i.e., products on the consumer market and in the case of ammonium nitrate, urea nitrate and urea in farming, while avoiding costly measures to track the products down to sales of small amounts.

To the extent the taggants can be applied directly at the source (initial manufacturing of a substance), it will also be an effective countermeasure against production of home-made explosives from stolen or illegally sold precursor materials for preparing explosives.

Taggants for fertilizers must be biodegradable, non-toxic, environmentally friendly, cheap, and easily detectable in low concentration. Also, a taggant should not affect the explosive properties of an explosive containing same.

The above-mentioned taggants used for conventional explosives are toxic, involving environmental hazard and therefore unsuitable for marking urea or ammonium nitrate, the latter being administered in large quantities in farming.

Tagging ammonium nitrate, urea nitrate and urea with non-volatile fluorescent molecules is advantageous only in cases where the explosive is visible and tangible. In contrast, volatile taggant can be detected by electronic sniffers and particularly by biosensors and animals, such as dogs and bees. The problem however is how to maintain long residence time of the volatile taggant within the bulk explosive.

Impregnation and coating of the spherical ammonium nitrate, urea nitrate and/or urea particles/crystals with the chosen volatile compound has several drawbacks as follows: (i) considering the huge quantities of ammonium nitrate, urea nitrate and/or urea that are produced daily worldwide the amounts of taggant needed to coat the bulk material would be prohibitively expensive; (ii) impregnation and coating of ammonium nitrate, urea nitrate and urea particles/crystals with lipophilic organic molecules would change the solubility properties of these fertilizers in wet soil, which would be commercially unacceptable by the consumers; (iii) the tagging procedure will require modifications of the production line, which may be unacceptable by the manufacturers.

For volatile taggants a mechanism of slow release of the volatile compound to the environment should be introduced. For example, the taggant layer can be protected by a second layer of a non-volatile matrix. However, coating the ammonium nitrate particle with two layers of insulating organic materials would further adversely affect the solubility properties of the fertilizer and may require major modifications of the production lines. Altogether, this approach seems unacceptable by both manufacturers and consumers.

The following publications, all of which I hereby incorporated by reference, describe different taggants and uses thereof.

U.S. Pat. No. 7,277,015 entitled “System and method for detecting, monitoring, tracking and identifying explosive materials” teaches a system and method for monitoring, detecting, tracking and identifying explosive materials. The system and method involves tracking and monitoring the explosive material during every part of the chain of custody.

U.S. Pat. No. 7,112,445 entitled “Fragmented taggant coding system and method with application to ammunition tagging” teaches identification tagging, and is specifically directed to identification tagging of ammunition. An isotopic taggant is deposited in a layer at the interface between the primer and the propellant so that, as the ammunition is fired, the taggant is dispersed throughout the propellant. The taggant is thus contained in the gunshot residue formed during the firing, and can be read by analysis of residue particles. Alternatively, the taggant may be deposited in a layer under the primer reactants, or in pellets which are easily destroyed by the chemical reactions involved in firing the ammunition, again dispersing the taggant throughout the propellant and the gunshot residue. Non-isotopic chemical taggants may also be employed if they are encoded so as to minimize the possibility of the information being destroyed or improperly read after the taggants are exposed to the chemical reactions in firing the ammunition. This is accomplished by employing a binary coding system and a system of authentication tags. Particulate taggants may also be used. The required large number of unique identification tags are obtained by using a fragmented coding system wherein each particle encodes only a portion of the serial number.

U.S. Pat. No. 6,025,200 entitled “Method for remote detection of volatile taggant” teaches a method of tagging and detecting objects is disclosed which comprises the steps of: (a) applying a volatile taggant to the object; and (b) subsequently detecting the presence of the taggant by the absorption, transmittance, reflectance, photon emission or fluorescence of the taggant and therefore a proximity of the tagged object. The present invention therefore provides optical sensing means which do not require physical separation of differing compounds for discrimination thereof.

WO/2008/138044A1 entitled “Explosive tagging” teaches a tagged explosive comprising an explosive composition and a precursor tag, the precursor tag comprising a transformable material that can transform during detonation of the explosive composition into a luminescent tag.

U.S. Pat. No. 3,897,284 entitled “Tagging explosives with organic microparticles” teaches tagging explosives by incorporating microparticles of a tack-free organic carrier, which microparticles have a distinctive geometric shape 1-250 micrometers in size. The microparticles contain tagging elements in uniform amounts of at least 0.1% of the total weight of each microparticle. By incorporating uniquely coded microparticles into each unit of production of explosive, any unit of production can be retrospectively identified by recovering and analyzing a single microparticle.

U.S. Pat. No. 4,053,433 entitled “Method of tagging with color-coded microparticles” teaches an improvement in the known method of tagging individual units of production of a substance with microparticles for retrospective identification is disclosed. The improvement comprises the use of microparticles which are encoded with an orderly sequence of visually distinguishable colored segments. Decoding of the microparticles can be accomplished with a microscope or other magnifying device.

U.S. Pat. No. 3,991,680 entitled “Tagging explosives with sulfur hexafluoride” teaches method and apparatus for tagging explosives with a source of SF₆ permitting the detection of their presence utilizing sensitive sniffing apparatus.

U.S. Pat. No. 4,131,064 entitled “Tagging particles which are easily detected by luminescent response, or magnetic pickup, or both” teaches small particles for tagging of objects to be identified comprise luminescent material plus other material which provides information indicia for tagging purposes. Included in the tagging particles are very small particles of magnetic material which is reflective both for the radiations which excite the luminescent material and also for the radiations generated by the excited luminescent material. The inclusion of the reflective magnetic particles introduces only a minimal decrease in the effectiveness of the luminescent material to provide a spotting or locating function. The particles may thus be located either by their luminescent response, or by magnetic pickup, or both. Inorganic species of these particles are especially useful for tagging explosives for post-explosion identification of the explosives.

Nayanhongo et al. (Nyanhongo G S, Aichernig N, Ortner M, Steiner W, and Guebitz G M, Journal of hazardous materials 2009, 165:285-90), teach the incorporation of TNT transforming bacteria in explosive formulations to facilitate biodegradation thereof if fails to detonate.

Additional background art includes U.S. Pat. Nos. 5,665,538 and 5,643,728.

Classical narcotics, such as, but not limited to heroin and opium are traditionally extracted from a variety of plants and are purified to a certain degree of purity. The entire manufacturing process of classical narcotics does not require a chemical industry facility.

Synthetic narcotics such as amphetamines and methamphetamine (N-methyl-1-phenyl-propan-2-amine or deoxyephedrine), which is an amine derivative of amphetamine (C₁₀H₁₅N) used in the form of its crystalline hydrochloride as a central nervous system stimulant, both medically and mostly illicitly. Methamphetamine increases alertness and energy, and in high doses, can induce euphoria, enhance self-esteem, and increase sexual pleasure. Methamphetamine has high potential for abuse, activating the psychological reward system by increasing levels of dopamine, norepinephrine and serotonin in the brain. Nicknames for methamphetamine are numerous and vary significantly from region to region. Some common nicknames for methamphetamine include “ice”, “meth”, “crystal”, “crystal meth”, “crank”, “glass”, “nazi dope”, “shabu” (Japan, Philippines, Malaysia), “tik” (South Africa), “ya ba” (Thailand), and “P” (New Zealand). Methamphetamine may also be referred to as “speed”, although this street term is officially reserved for regular amphetamine, without the methyl group.

Typically, the synthesis of synthetic narcotics requires at least one ingredient which is legitimately manufactures by the chemical industry. Indeed, in the case of methamphetamines, ephedrine or pseudoephedrine, both are active pharmaceutical ingredients (APIs) in nasal decongestive medicaments, such as SUDAFED, are not simple for synthesis and are produced by less than a dozen chemical production facilities globe wide.

### SUMMARY OF THE INVENTION

The present invention relates to taggants and uses of taggants to tag materials, such as explosives and substances used in their preparation.

According to an aspect of some embodiments of the present invention there is provided a composition of matter which comprises a non-gaseous nitrogen containing compound; and a spore of a micro-organism.

According to an aspect of some embodiments of the present invention there is provided a composition of matter which comprises an explosive; and a spore of a micro-organism.

According to an aspect of some embodiments of the present invention there is provided a composition of matter which comprises a substance used in the preparation of an explosive; and a spore of a micro-organism.

According to an aspect of some embodiments of the present invention there is provided a method of determining a source of a material the material being an explosive or a substance used in the preparation of the explosive, the material comprises a spore of a micro-organism uniquely associated with the material, the method comprising inducing the spore to transform into a vegetative micro-organism and identifying the micro-organism as being associated with the material, thereby determining the source of the material.

According to an aspect of some embodiments of the present invention there is provided a taggant composition comprising a spore of a micro-organism and a carrier, the carrier for distributing the taggant in larger volumes.

According to an aspect of some embodiments of the present invention there is provided a method of tagging a material, the method comprising mixing the material with a spore of a micro-organism.

According to an aspect of some embodiments of the present invention there is provided a composition of matter comprising (a) a substance used in the preparation of a narcotic; and (b) a spore of a micro-organism.

According to an aspect of some embodiments of the present invention there is provided a composition of matter comprising (a) a narcotic; and (b) a spore of a micro-organism.

According to an aspect of some embodiments of the present invention there is provided a composition of matter comprising (a) a substance used in the synthesis of a narcotic; and (b) a spore of a micro-organism.

According to an aspect of some embodiments of the present invention there is provided a method of determining a source of a material the material being a narcotic or a substance used in the preparation of the narcotic, the material comprises a spore of a micro-organism uniquely associated with the material, the method comprising inducing the spore to transform into a vegetative micro-organism and identifying the micro-organism as being associated with the material, thereby determining the source of the material.

According to an aspect of some embodiments of the present invention there is provided a taggant composition comprising a spore of a micro-organism and a carrier, said carrier for distributing said taggant in larger volumes, said carrier is at a pharmaceutical grade.

According to an aspect of some embodiments of the present invention there is provided a method of tagging ephedrine or pseudoephedrine, the method comprising mixing the ephedrine or pseudoephedrine with a spore of a micro-organism.

According to some embodiments of the invention, the micro-organism cannot degrade an explosive.

According to some embodiments of the invention, the micro-organism cannot degrade 2,4,6-trinitrotoluene.

According to some embodiments of the invention, the micro-organism is a bacteria.

According to some embodiments of the invention, the micro-organism is archaea.

According to some embodiments of the invention, the micro-organism is a eukaryote.

According to some embodiments of the invention, the micro-organism is an algea.

According to some embodiments of the invention, the micro-organism is a fungi.

According to some embodiments of the invention, the micro-organism is an extremophiles, such as a thermophile, psychrophile, acidophile, alkaliphile, halophile.

According to some embodiments of the invention, the micro-organism is an auxotroph.

According to some embodiments of the invention, the auxotroph is selected from the group consisting of a nucleotide (e.g., uracil) dependent auxotroph, an amino acid dependent auxotroph, a fatty acid dependent auxotroph, a vitamin dependent auxotroph.

According to some embodiments of the invention, the micro-organism has a resistivity to an antibiotic.

According to some embodiments of the invention, the antibiotic is selected from the group consisting of beta-lactam antibiotics, aminoglycoside antibiotics and and polyketide antibiotics.

According to some embodiments of the invention, the antibiotic is selected from the group consisting of ampicillin, kanamycin and tetracycline.

According to some embodiments of the invention, the micro-organism includes a DNA barcode.

According to some embodiments of the invention, the DNA barcode encodes for at least one information selected from the group consisting of manufacturer, type of material, production date, production plant and ordering information.

According to some embodiments of the invention, the composition is a solid. According to some embodiments of the invention, the nitrogen containing compound is a nitro (—NO₂) containing compound.

According to some embodiments of the invention, the nitrogen containing compound is antiroaromatic compound.

According to some embodiments of the invention, the nitrogen containing compound is a nitramine (—N—NO₂) containing compound.

According to some embodiments of the invention, the nitrogen containing compound is a nitrate ester (—O—NO₂) containing compound.

According to some embodiments of the invention, the nitrogen containing compound is an amino group containing compound.

According to some embodiments of the invention, the nitrogen containing compound is a diazo group (—N═N—) or triazo group (—N═N—N═) containing compound.

According to some embodiments of the invention, the nitro compound is a fertilizer.

According to some embodiments of the invention, the fertilizer comprises ammonium nitrate

According to some embodiments of the invention, the nitrogen containing compound is urea.

According to some embodiments of the invention, the nitro compound is an explosive.

According to some embodiments of the invention, the explosive is selected from the group consisting of NT 2-nitrotoluene; NT 3-nitrotoluene; NT 4-nitrotoluene; TNT 2,4,6-trinitrotoluene; DNT 2,4-dinitrotoluene; DNT 3,4-dinitrotoluene; DNT 2,6-dinitrotoluene; EGDN ethylene glycol dinitrate; NG nitroglycerine; RDX cyclotrimethylenetrinitramine (cyclonite); PETN pentaerythritol tetranitrate; HMX homocyclonite (octogen); NH4NO3 Ammonium nitrate; NitroBid 1,2,3-propanetrial trinitrate Formulations; C-4 RDX and/or PETN Semtex RDX and/or PETN Detasheet RDX and/or PETN Dynamites EDGN and/or NG.

According to some embodiments of the invention, the explosive is prior to its blast.

According to some embodiments of the invention, the explosive is post blast.

According to some embodiments of the invention, wherein the material is a substance used in the preparation of an explosive.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described herein. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a cross sectional exemplary view of a bacterial endospore showing its core, coat, outer membrane, cortex, germ cell wall and inner membrane.

### DESCRIPTION OF EMBODIMENTS OF THE INVENTION

The present invention, in some embodiments thereof, relates to a taggant and its uses, more particularly, but not exclusively, to (i) a taggant incorporated in explosives and in substances used in the preparation of explosives, (ii) explosives and substances used in the preparation of explosives incorporating the taggant; (iii) methods for their production; and (iv) methods of identifying the source of an explosive, before or after detonation or blast, and (v) the source of substances used in the preparation of explosives.

Before explaining at least one embodiment of the invention in detail, it is to be understood that the invention is not necessarily limited in its application to the details set forth in the following description or exemplified by the examples. The invention is capable of other embodiments or of being practiced or carried out in various ways.

As used herein in the specification and in the claims section below, the term “spore” refers to a reproductive or proliferative unit of a micro-organism. Spores are known to be resistant to stresses including a-biotic stresses such as heat, hostile chemical environment, dryness and the like. Specific spores include endospores and cysts.

As used herein in the specification and in the claims section below, the term micro-organism,” refers to an organism that is typically microscopic (too small to be seen by the naked human eye) and/or unicellular. Microorganisms are very diverse; they include bacteria, fungi, archaea, and protists; microscopic plants (called green algae); and animals such as plankton, the planarian and the amoeba. Most microorganisms are unicellular (single-celled), but this is not universal, since some multicellular organisms are microscopic, while some unicellular protists and bacteria, like Thiomargarita namibiensis, are macroscopic and visible to the naked eye.

Micro-organisms sense and adapt to changes in their environment. When favored nutrients are exhausted, some micro-organisms (e.g., certain bacteria) may become motile to seek out nutrients, or they may produce enzymes to exploit alternative resources. One example of an extreme survival strategy employed by micro-organisms is the formation of spores. An exemplary spore is the bacterial endospore form in a process known as endospore sporulation. This complex developmental process is often initiated in response to nutrient deprivation. It allows the bacterium to produce a dormant and highly resistant structure to preserve the cell's genetic material in times of extreme stress.

Spores in general and endospores in particular can survive environmental assaults that would normally kill a micro-organism. These stresses include high temperature, high UV irradiation, desiccation, chemical damage and enzymatic destruction. The extraordinary resistance properties of spores and endospores make them of particular importance because they are not readily killed by many harsh antimicrobial treatments.

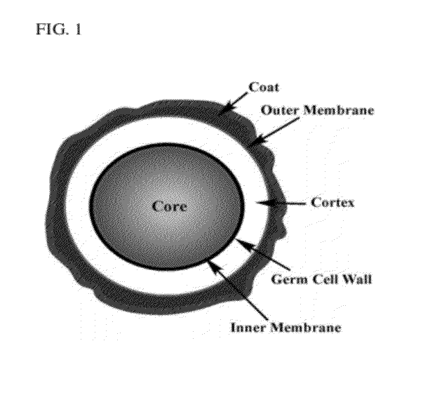

Many organisms form spores. The bacterial endospore is unique in its heat resistance capabilities resulting from its multilayer structure, as shown in FIG. 1 (see also In H. H. Hyun et al., Ultrastructure and Extreme Heat Resistance of Spores from Thermophilic Clostridium Species. J. Bacteriol. 1983, 156, 1332-1337):

Exosporium—a thin delicate covering made of protein.

Spore coats—composed of layers of spore specific proteins.

Cortex—composed of loosely linked peptidoglycan and contains dipicolinic acid (DPA), which is particular to all bacterial endospores. The DPA cross links with calcium ions embedded in the spore coat. This cross linkage greatly contributes to the extreme resistance capabilities of the endospores because it creates a highly impenetrable barrier. The calcium DPA cross linkages compose 10% of the dry weight of the endospores.

Core—the core contains the usual cell wall and, cytoplasmic membrane, nucleoid, and cytoplasm. The core only has 10-30% of the water content of vegetative cells; therefore the core cytoplasm is in a gel state. The low water content contributes to the endospores success in dry environments. However, the low water concentration and gel cytoplasm contributes to the inactivity of cytoplasmic enzymes. The core cytoplasm is also one unit lower in pH than the vegetative cell, thus conferring acidic environment survival. SASPs, small acid soluble spore proteins, are formed during sporulation and bind to DNA in the core. SASPs protect the DNA from UV light, desiccation, and dry heat. SASPs also serve as a carbon energy source during germination, the process of converting a spore back to a vegetative cell.

According to an aspect of some embodiments of the present invention, there is provided a composition of matter which comprises a non-gaseous nitrogen containing compound and further comprising a spore of a micro-organism.

According to another aspect of some embodiments of the present invention there is provided a composition of matter which comprises an explosive; and a spore of a micro-organism.

According to another aspect of some embodiments of the present invention there is provided a composition of matter which comprises a substance used in the preparation of an explosive; and a spore of a micro-organism.

According to another aspect of some embodiments of the present invention there is provided a method of determining a source of a material the material being an explosive or a substance used in the preparation of the explosive, the material comprises a spore of a micro-organism uniquely associated with the material, the method comprising inducing the spore to transform into a vegetative micro-organism and identifying the micro-organism as being associated with the material, thereby determining the source of the material.

According to another aspect of some embodiments of the present invention there is provided a taggant composition comprising a spore of a micro-organism and a carrier, the carrier for distributing the taggant in larger volumes.

According to an aspect of some embodiments of the present invention there is provided a method of tagging a material, the method comprising mixing the material with a spore of a micro-organism. In this method a taggant composition may be employed as is further delineated hereinbelow.

According to some embodiments of the invention, the micro-organism cannot degrade an explosive, the explosive may be any one or more of the explosives listed herein, 2,4,6-trinitrotoluene in particular.

The micro-organism used in context of the present invention is a prokaryote, namely, bacteria or archea, or a eukaryote, namely protists, animals, fungi and plants (e.g., algae), provided the micro-organism can sporulate into spores.

According to some embodiments of the invention, the micro-organism is an extremophile.

As used herein in the specification and in the claims section that follows, the term “extremophile” refers to micro-organisms which are capable of growing under unusual, thus extreme, environmental conditions. Extremophiles come in all kinds of forms and from all kingdoms. Unless sporolated, many of the extremophiles cannot survive under what one calls, from an anthropogenic point of view, ‘normal conditions’, i.e., conditions conducive to the human physiology and for most of the micro-organisms associated with humans, which are mesophilic, neutrophilic micro-organisms, whereas mesophilic neutrophilic micro-organisms cannot grow in environments in which extremophiles thrive.

Micro-organisms preferring extreme conditions include, but are not limited to, heat-loving, i.e., hyper (Tₒₚₜ above 80° C.), e.g., Methanopyrus kandleri strain 116 and 121, Pyrolobus fumarii, Pyrococcus furiosus, all Archaea and Geothermobacterium ferrireducens and Aquifex aeolicus, both bacteria, extreme (Tₒₚₜ above 65° C.) and thermophiles (Tₒₚₜ above 55° C.), e.g., Thermus aquaticus, Thermococcus litorali, Clostridium thermocellum LQRI, Clostridium thermosulfurogenes 4B, and Clostridium thermohydrosulfuricum 39E; cold-loving, i.e., psychrophiles (Tₒₚₜ<15° C.), e.g., Chromobacterium spp., Brevibacterium spp. and Brochothrix spp., Pseudomonas, Vibrio, Acinetobacter/Alcaligenes, Aeromonas, Chromobacterium, Flavobacterium, Serratia, Yersinia, Arthrobacter, Corynebacterium, Brevibacterium, Cellulomonas, Lactobacillus, Brochothrix, Streptococcus, Micrococcus, Listeria, Bacillus, Clostridim, Cytophaga, Fragillaria, Chlamydomonas, Raphidonema, Chloromanas, Cylindrocystis, Candida, Typhula, Leptomitus, Mucor, Rhizopus, Penicillium, Alternaria, Cladosporium, Keratinomyces; acid- or alkaline-loving, i.e., acidophiles (pHₒₚₜ<5.0), e.g., Sulfolobales, Crenarchaeota, Thermoplasmatales, ARMAN, Acidianus brierley, Metallosphaera sedula, thermoacidophilic, all of which are Archea and Acidobacterium, Acidithiobacillales, ferrooxidans, thiooxidans, Thiobacillus prosperus, Thiobacillus acidophilus, Thiobacillus organovorus, Thiobacillus prosperus Thiobacillus cuprinus, Acetobacter aceti and Nocardiopsis alba, Sulfolobales, Thermoplasmatales, and Metallosphaera sedula all of which are bacteria, and alkaliphiles (pHₒₚₜ>8.0), e.g., Geoalkalibacter ferrihydriticus, Bacillus okhensis and Alkalibacterium iburiense, respectively; salt-loving, i.e., halophiles, such as Halobacterium, Halococcus, Nitzschia, Bacillariaceae, Lovenula, Diaptomidae, Salinibacter ruber, Dunaliella salina, Haloferax, Halogeometricum, Halococcus, Haloterrigena, Halorubrum, Haloarcula and Halobacterium; extremely low-substrate-concentration-preferring or -requiring, i.e., oligophiles or ‘oligotrophs’; extremely high-substrate-concentration-preferring or -requiring, i.e., ‘copiotrophs’; high-pressure-loving, i.e., barophiles or piezophiles. Also, micro-organisms growing at high concentrations of heavy metal ions, under high doses of gamma and UV radiation, high solvent concentrations or very low-water-activity conditions, i.e., dry-resistant micro-organisms from the desert, are regarded as extremophiles. Micro-organisms that can grow extremely fast with doubling times below 15 minutes ('hyperauxanophiles' i.e., significantly faster than Escherichia coli) are also regarded as extremophiles. Examples include some of the alkalithermophiles with doubling times around 10 minutes or the neutrophilic marine mesophile Vibrio natriegans with doubling times below 10 minutes. In other words what is ‘normal’ for E. coli and similar micro-organisms is extreme for the extremophiles and vice versa.

Other extremophiles include, but are not limited to, capnophiles which are extremophiles which thrive in the presence of high concentrations of carbon dioxide, or which require the presence of carbon dioxide to survive; endoliths, which are extremophiles which that live inside rock, coral, animal shells, or in the pores between mineral grains of a rock; hypoliths which are photosynthetic extremophile organisms that live underneath rocks in climatically extreme deserts; lipophile, which are which are extremophiles which can thrive in an oily environment, lithoautotrophs which are extremophiles which derive energy from reduced compounds of mineral origin; lithophile which are extremophiles which that can live within the pore spaces of sedimentary and even igneous rocks to depths of several kilometers, osmophiles which are extremophiles which that are able to grow in environments with a high sugar concentration, e.g., Saccharomyces rouxii, Saccharomyces bailii, Debaryomyces and Saccharomyces cerevisiae, and xerophiles are extremophiles that can grow and reproduce in conditions with a low availability of water, also known as water activity, e.g., Trichosporonoides nigrescens and Cacti.

Polyextremophile are extremophiles which combines at least two extremophilic features, e.g., thermoacidophiles.

Notable extremophiles not mentioned above include, without limitation, Chloroflexus aurantiacus, Deinococcus radiodurans, Deinococcus-Thermus, Paralvinella sulfincola, Pompeii worm, Pyrococcus furiosus, Snottite Strain 121, Spirochaeta americana and Tardigrada.

Examples of anaerobic alkalithermophiles, facultative and aerobic alkalithermophiles and thermophilic fungi are provides in Examples 1-3 of the Examples section below.

According to some embodiments of the invention, the micro-organism is an auxotroph. Auxotrophy is the inability of an organism to synthesize a particular organic compound required for its growth. An auxotroph is an organism that displays this characteristic Auxotrophy is the opposite of prototrophy. In genetics and biology, a strain is said to be auxotrophic if it carries a mutation that renders it unable to synthesize an essential compound. For example a yeast mutant in which a gene of the uracil synthesis pathway is inactivated is a uracil auxotroph. Such a strain is unable to synthesize uracil and will only be able to grow if uracil can be taken up from the environment. This is the opposite of a uracil prototroph, or in this case a wild-type strain, which can still grow in the absence of uracil. Auxotrophic genetic markers are often used in molecular genetics; they were famously used in Beadle and Tatum's Nobel prize-winning work on the one gene-one enzyme hypothesis. Researchers have used strains of E. coli auxotrophic for specific amino acids to introduce non-natural amino acid analogues into proteins. For instance cells auxotrophic for the amino acid phenylalanine can be grown in media supplemented with an analogue such as para-azido phenylalanine.

Thus, according to some embodiments of the invention, the auxotroph is selected from the group consisting of a nucleotide dependent auxotroph, an amino acid dependent auxotroph, a fatty acid dependent auxotroph, a vitamin dependent auxotroph. In this context, the nucleotide can be, adenosine, guanine, cytosine, thymine, uracil, deoxiadenosine, deoxiguanine, deoxicytosine and deoxithymine; the amino acid can be histidine, isoleucine, leucine, lysine, methionine, phenylalanine, threonine, tryptophan, valine, alanine, arginine, aspartic acid, cysteine, cystine, glutamic acid, glutamine, glycine, hydroxyproline, ornithine, proline, serine and tyrosine, the vitamin and/or their precursors can be vitamin A (carotene), vitamin D (ergocalciferol and cholecalciferol), vitamin E (tocopherol), vitamin K (phylloquinone; menaquinone), vitamin B1 (thiamin), vitamin B2 (riboflavin), vitamin B6 (pyridoxine), vitamin B12 (cobalamin), niacin (nicotinic acid and nicotinamide), pantothenic acid, biotin, folic acid, vitamin C (ascorbic acid).

According to some embodiments of the invention, the micro-organism has a resistivity to an antibiotic. The antibiotic can be any one or more of beta-lactam antibiotics, aminoglycoside antibiotics and polyketide antibiotics, including, but not limited to, ampicillin, kanamycin and tetracycline, respectively.

Additional antibiotics include atovaquone, cephalosporins, clindamycin, aztreonam, cefepime, ceftazidime, ciprofloxacin, clindamycin, gentamicin, metronidazole, pentamidine, pyrimethamine, sulfadiazine, SMZ-TMP, trimethoprim, tetracycline, vancomycin, ciprofloxacin, metronidazole, pyrimethamine, metronidazole, pentamidine, pyrimethamine, sulfadiazine and trimethoprim.

According to some embodiments of the invention, the micro-organism includes a DNA barcode.

According to some embodiments of the invention, the DNA barcode encodes for at least one information selected from the group consisting of manufacturer, type of material, production date, production plant and ordering information.

By selecting the micro-organism for use in context of the present invention to be extremophiles, auxotrophs, having antibiotic resistant and/or coded with a DNA barcode, one allows to tag with a readily identifiable micro-organism being at its most durable and resilient form (i.e., sporolated into a spore) any material tagged therewith, including, without limitation, explosives and substances which may be used in their production, inclusive, as is further described herein, of certain fertilizers.

For efficient use of the invention, a database, e.g., a computer database, may be generated, and may have public or law-enforcement agencies restricted access, in which: (a) data pertaining to a taggant, i.e., a micro-organism or a predetermined combination of micro-organisms (e.g., extremophiles, auxotrophs, antibiotic resistant and/or coded with a DNA barcode, the spores thereof serve as the taggant), which is mixed with a substance to be subsequently monitored, including, without limitation, the name of the micro-organism(s), their combination, conditions for their optimal and/or differential growth and the like, is associated with (b) data pertaining to one or more of the following details relating to the substance and its source: details relating to the type of substance, details relating to the batch or date of its production, details relating to its manufacturer, details relating to the buyer or the order placer of the substance, details relating to the shipment of the substance, etc.

When a source of a substance is to be determined, the substance is characterized for its spores content, using microbiological, biochemical and/or genetic techniques and the spores content (i.e., name of organism) can be searched for in the database and the type and source of the substance may thus be identified.

For example, conditions for characterizing extremophiles may include growth under extreme (hot or cold) temperatures, extreme pH (acidic or alkilic), extreme salt concentration, extreme nutrient concentration, low water avidity and so on, all as is exemplified herein. Extremophiles are advantageous in this context because under the conditions in which they may grow and thrive mesophilic, neutrophilic micro-organisms cannot grow.

Conditions for characterizing an auxotroph would include conditions in which the essentiality of a certain chemical compound to the growth of the organism is tested and determined. Such conditions may include parallel testing for growth of micro-organism isolates with and without the essential compound, examples of essential compounds are provided hereinabove.

Conditions for characterizing resistivity to an antibiotic or a combination of antibiotics include growth in the presence of the antibiotic or the combination thereof.

Conditions for characterizing a DNA barcode of a micro-organism include genetic characterization of the presence and informational content (nucleotide sequence) of the DNA barcode. U.S. Pat. Nos. 7,510,829, 7,460,223, 7,323,309, 7,262,032, 7,187,286, 6,974,669, 6,858,412, 6,677,121, 6,403,319, 6,383,754 and 6,261,782, all of which are incorporated herein by reference, provide a short list of references that teach how to create and use DNA barcodes, which are also known in the art as genetic barcodes or tags or DNA tags. The DNA sequence information carried by the DNA barcode may include information relating to, such as, but not limited to, a manufacturer, it address and contacting information, a type of substance, a production date/batch, production plant, ordering information, buyer, etc. DNA barcodes are suitable in this context because when prepared synthetically and inserted into a genome of an organism, they may acquire any desired sequence and may encode any amount of information, using e.g., a universal alphanumeric or binary coding system. These sequences are readily identifiable and sequenced using amplification and sequencing techniques such as PCR and automated DNA sequencing.

As is stated above, a combinatorial approach for tagging substances may be adopted for practicing the present invention, namely, different combinations of spores of two or more (e.g., 3, 4 5, 6, 7, 8, 9, 10, 11-20, 21-100 or more) different micro-organisms may be used to tag different substances. This approach entails that more that one growth conditions may be employed in order to identify the type of the micro-organisms, the spores thereof are present in a tested sample.

All strains of micro-organisms in general and strains having antibiotic resistance in particular, may be selected so as not to be pathogenic to humans and organisms residing in their vicinity.

As is apparent to the skilled artisan, the practice of the present invention employs conventional techniques of molecular biology (including recombinant techniques, amplification techniques and sequencing techniques), microbiology and biochemistry, which are within the skill of art practitioners. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); and Short Protocols in Molecular Biology (Wiley and Sons, 1999).

According to some embodiments of the invention, the nitrogen containing compound is a nitro (—NO2) containing compound, antiroaromatic compound, a nitramine (—N—NO2) containing compound, a nitrate ester (—O—NO2) containing compound, an amino group containing compound a diazo group (—N═N—) or triazo group (—N═N—N═) containing compound and/or urea.

According to some embodiments of the invention, the nitro compound is a fertilizer. According to some embodiments of the invention, the fertilizer comprises or is ammonium nitrate. According to some embodiments of the invention, the nitro compound is an explosive. According to some embodiments of the invention, the explosive is selected from the group consisting of NT 2-nitrotoluene; NT 3-nitrotoluene; NT 4-nitrotoluene; TNT 2,4,6-trinitrotoluene; DNT 2,4-dinitrotoluene; DNT 3,4-dinitrotoluene; DNT 2,6-dinitrotoluene; EGDN ethylene glycol dinitrate; NG nitroglycerine; RDX cyclotrimethylenetrinitramine (cyclonite); PETN pentaerythritol tetranitrate; HMX homocyclonite (octogen); NH4NO3 Ammonium nitrate; NitroBid 1,2,3-propanetrial trinitrate Formulations; C-4 RDX and/or PETN Semtex RDX and/or PETN Detasheet RDX and/or PETN Dynamites EDGN and/or NG.

The methods of the present invention may be practiced to identify spores of micro-organisms as described herein in any substance, including, ad detailed herein, explosives and substances used in their production. Spores present in an explosive may be spores that were added to the explosive itself or spores previously added to one of substances used in the preparation of the explosive prior to the use of the substance in the preparation of the explosive. Either way, spores present in an explosive may be identified and hence the source of the explosive or the substances used in its production identified also post blast. This is due do to (a) the resilient nature of spores which can withstand harsh conditions, such harsh chemical conditions and even blast; an (b) the inherent advantage of using a proliferating objects (i.e., spores) that once induced to proliferate multiplies exponentially and hence allow their detection and characterization even if one unit thereof is present in a tested sample.

In some embodiments of the invention the amount of spores of a single micro-organism species mixed into a gram of material, e.g., an explosives or a substance used in the preparation if an explosive or a carrier, are at least 103, alternatively, at least 104, alternatively, at least 105, alternatively, at least 106, alternatively, at least 107, alternatively, at least 108, alternatively, at least 109, alternatively, at least 1010, alternatively, at least 1011, alternatively, at least 1012, alternatively, at least 1013, alternatively, at least 1014, alternatively, at least 1015, alternatively, at least 1016, alternatively, at least 1017, alternatively, at least 1018, alternatively, at least 1019, alternatively, at least 1020-1030 or more.

The carrier, which is used according to the present invention to facilitate the distribution of spores in larger volumes may be, for example, a substance having high mixability and high pourability properties, as these properties apply to the art of mixing dry substances. The carrier may be chemically inert in that it does not interfere in the process of preparing an explosive and/or the blast of an explosive.

Hence, according to some aspects and embodiments of the present invention, there are provided (i) a taggant incorporated in explosives and in substances used in the preparation of explosives, (ii) explosives and substances used in the preparation of explosives incorporating the taggant; (iii) methods for their production; and (iv) methods of identifying the source of an explosive, before or after detonation or blast, and (v) the source of substances used in the preparation of explosives.

A taggant composition according to the invention may include spores of at least one micro-organism as herein described and a carrier, the carrier is for assisting to achieve homogenous distribution of the spores in large volumes of material. Techniques for adequate and homogenous mixing of materials are well known in the explosive, pharmaceutical, powdered food and fertilizer production industries and are hence apparent to the skilled artisan.

It is expected that during the life of a patent maturing from this application many relevant spores and explosives will be developed and the scope of the respective terms is intended to include all such new technologies a priori.

When illicitly produced, methamphetamine is commonly made by the reduction of ephedrine or pseudoephedrine. Most of the necessary chemicals are readily available in household products or over-the-counter cold or allergy medicines. Synthesis is relatively simple. Most methods of illicit production of methamphetamine involve protonation of the hydroxyl group on the ephedrine or pseudoephedrine molecule. The most common method for small-scale methamphetamine labs in the United States is primarily called the “Red, White, and Blue Process”, which involves red phosphorus, pseudoephedrine or ephedrine (white), and blue iodine (which is technically a purple color in elemental form), from which hydroiodic acid is formed. In Australia, criminal groups have been known to substitute “red” phosphorus with either hypophosphorous acid or phosphorous acid. Another common method uses the Birch reduction (also called the “Nagai method”), in which metallic lithium, commonly extracted from non-rechargeable lithium batteries, is substituted for difficult-to-find metallic sodium. The Birch reduction is dangerous because the alkali metal and liquid anhydrous ammonia used in the process are both extremely reactive, and the temperature of liquid ammonia makes it susceptible to explosive boiling when reactants are added. Anhydrous ammonia and lithium or sodium (Birch reduction) may be surpassing hydroiodic acid (catalytic hydrogenation) as the most common method of manufacturing methamphetamine in the U.S., and possibly in Mexico.

A completely different procedure of synthesis uses the reductive amination of phenylacetone with methylamine, both of which are currently DEA list I chemicals (as are pseudoephedrine and ephedrine). The reaction requires a catalyst that acts as a reducing agent, such as mercury-aluminum amalgam or platinum dioxide, also known as Adams' catalyst. This was once the preferred method of production by motorcycle gangs in California, until DEA restrictions on the chemicals made the process difficult. Other less common methods use other means of hydrogenation, such as hydrogen gas in the presence of a catalyst.

Hence, for the sake of monitoring the source of substances used in the preparation of narcotics, such as, but not limited to, methamphetamine, according to an aspect of some embodiments of the present invention there is provided a composition of matter comprising (a) a substance used in the preparation of a narcotic; and (b) a spore of a micro-organism.

As used herein the term “narcotic” refers to an addictive substance, that when administered to a human it reduces pain and/or alters mood and behavior.

According to an aspect of some embodiments of the present invention there is provided a composition of matter comprising (a) a narcotic; and (b) a spore of a micro-organism.

According to an aspect of some embodiments of the present invention there is provided a composition of matter comprising (a) a substance used in the synthesis of a narcotic; and (b) a spore of a micro-organism.

According to an aspect of some embodiments of the present invention there is provided a method of determining a source of a material the material being a narcotic or a substance used in the preparation of the narcotic, the material comprises a spore of a micro-organism uniquely associated with the material, the method comprising inducing the spore to transform into a vegetative micro-organism and identifying the micro-organism as being associated with the material, thereby determining the source of the material.

According to an aspect of some embodiments of the present invention there is provided a taggant composition comprising a spore of a micro-organism and a carrier, said carrier for distributing said taggant in larger volumes, said carrier is at a pharmaceutical grade, e.g., acceptable for use in a pharmaceutical composition by the pharmaceutical industry.

According to an aspect of some embodiments of the present invention there is provided a method of tagging ephedrine or pseudoephedrine, the method comprising mixing the ephedrine or pseudoephedrine with a spore of a micro-organism.

Other aspects and embodiments which are described herein in context of explosives or substances used in their preparation (e.g., types of spores, etc.) can be similarly used in context of narcotics and substances used in their preparation and/or synthesis.

The terms “comprises”, “comprising”, “includes”, “including”, “having” and their conjugates mean “including but not limited to”. These terms encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, biological, microbiological and biochemical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Reference is now made to the following examples, which together with the above descriptions, illustrate some embodiments of the invention in a non limiting fashion.

### Example 1

TABLE 1
Exemplary anaerobic alkalithermophiles

| | | Determined | |
|---|---|---|---|
| Species | pHₒₚₜ | at ° C. | Tₒₚₜ (° C.) |
| Clostridium paradoxum | 10.3 | 55 | 54-58 |
| Clostridium thermoalcaliphilum | 9.8 | 50 | 49 |
| Anaerobranca gottschalkii | 9.5 | n | 53 |
| ‘Thermopallium natronophilum’ | 9.5 | n | 70 |
| Anaerobranca sp. KS5Y | 8.7 | n | 57 |
| Thermosyntropha lipolytica | 8.5 | 25 | 63 |
| Desulfotomaculum alkaliphilum | 8.6 | n | 53 |
| Anaerobranca horikoshii | 8.5 | 60 | 57 |
| Halonatronum saccharophilum | 8.3 | n | 46 |
| Thermobrachium celere | 8.2 | 60 | 65-67 |
| Caloramator indicus | 8.1 | n | 63 |
| Thermococcus acidaminovorans | 9 | n | 85 |
| Thermococcus alkaliphilus | 9 | n | 85 |
| Thermococcus fumicolans | 8.5 | n | 85 |
| Methanothermobacter | 8 | n | 60 |
| thermautotrophicus AC60 | | | |
| Methanothermobacter thermoflexus | 8 | n | 55 |

n, not known;
Tₒₚₜ, temperature optimum for growth rate.

### Example 2

TABLE 2
Exemplary facultative and aerobic alkalithermophiles

| | | pH | Tₒₚₜ | Enzymes |
|---|---|---|---|---|
| Species | Comments | (range) | (° C.) | produced |
| Bacillus alcalophilus | Tolerates up | 10.6 | 60-65 | Extracellular |
| B-M20 | to 7.5% | (8-12) | | lipase (pH 10.6 |
| | NaCl | | | and 60° C.) |
| Thermoactinomyces | Tolerates up | 10.3 | 50 | Extracellular |
| sp. HS682 | to 10% NaCl | (7.5-11.5) | | serine protease. |
| | | | | Purified: 11.5 |
| | | | | and 70 |
| Geobacillus | | 10.0 | 60 | Purified: 9.0 |
| stearothermophilus | | (ND) | | and 75° C. |
| F1 | | | | Extracellular |
| | | | | protease |
| Anoxybacillus | Reduces | 9.5-9.7 | 62 | |
| pushchinoensis KIT | nitrate to | (8.0-10.5) | | |
| | nitrite | | | |
| Bacillus halodurans | 1 out of 16 | 9-10 | 55 | Has been |
| | strains; | (ND) | | studied in |
| | tolerates up | | | terms of |
| | to 12% NaCl | | | systematic |
| | | | | position |
| Bacillus sp. TA2-A1 | 100 mM | 9.2 | 70 | Has been |
| | NaCl is | (7.7-10.5) | | studied in |
| | required | | | terms of |
| | | | | bioenergetics |
| Sphaerobacter | | 8.5 | 55 | |
| thermophilus S6022^{T} | | (ND) | | |
| Bacillus | | 8-9 | 55 | |
| thermocloaceae | | (7-?) | | |
| S6025^{T} | | | | |
| Thermomicrobium | Contains | 8.2-8.5 | 70-75 | |
| roseum ATCC | carotenoids | (6.0-9.4) | | |
| 27502^{T} | | | | |
| Bacillus pallidus | At 10% | 8.0-8.5 | 60-65 | |
| H12^{T} | NaCl | (ND) | | |
| Bacillus ‘caldotenax’ | | 7.5-8.5 | 80 | Extracellular |
| YT G | | (ND) | | amylase, |
| | | | | alkaline |
| | | | | phosphatase, |
| | | | | protease: |
| | | | | 70° C. |
| Anoxybacillus | | 6.8-8.5 | 57-62 | |
| ‘kamchatkensis’ | | (5.7-9.9) | | |
| KG4^{T} | | | | |
| Thermoactinomyces | | 8.5 | 50 | Cellulase free |
| sacchari A-1 | | (ND) | | extracellular |
| | | | | endo-1,4-b- |
| | | | | xylanase (EC |
| | | | | 3.2.1.8) |

Tₒₚₜ, temperature optimum for growth rate.

### Example 3

TABLE 3
Taxonomic status and cardinal temperatures of thermophilic fungi^{a}

| Fungus (present | | T_{Opt} | Tₘₐₓ |
|---|---|---|---|
| nomenclature) | Other names | (° C.) | (° C.) |
| Canariomyces | | 45 | |
| thermophila Guarro & | | | |
| Samson | | | |
| Chaetomium | | 45 | 52 |
| mesopotamicum | | | |
| Abdullah & Zora | | | |
| Chaetomium | C. thermophilum, C. | 45-55 | 58-61 |
| thermophile La Touche | thermophilium | | |
| Coonemeria aegyptiaca | Thermoascus aegyptiacus, | 40 | 55 |
| (Ueda & Udagawa) | Paecilomyces aegyptiaca | | |
| Mouchacca | | | |
| Coonemeria crustacea | Thermoascus crustaceus, | 40 | <60 |
| (Apinis & Chesters) | Dactylomyces crustaceus, | | |
| Mouchacca | Paecilomyces crustaceus | | |
| Coonemeria verrucosa | Thermoascus crustaceus | 30-40 | 55 |
| (Yaguchi, Someya et | | | |
| Udagawa) Mouchacca | | | |
| Corynascus | Thielavia thermophila, | 50 | 60 |
| thermophilus (Fergus & | Myceliophthora fergusii, | | |
| Sinden) van Klopotek | Chrysosporium fergusii | | |
| Dactylomyces | Thermoascus thermophilus, | 40-45 | |
| thermophilus Sopp | Thermoascus aurantiacus | | |
| | (misapplied name) | | |
| Malbranchea | Trichothecium | 45 | 57 |
| cinnamomea (Libert) | cinnamomeum, | | |
| van Oorschot & de | Thermoidium sulfureum, | | |
| Hoog | Malbranchea pulchella | | |
| | var. sulfurea | | |
| Melanocarpus | Myriococcum albomyces, | 45 | 57 |
| albomyces (Cooney & | Thielavia albomyces | | |
| Emerson) von Arx | | | |
| Melanocarpus | Thielavia minuta var. | 35 | 50 |
| thermophilus (Abdullah | thermophila | | |
| & Al-Bader) Guarro, | | | |
| Abdullah & Al-Bader | | | |
| Myceliophthora | | 40-45 | >50 |
| hinnulea Awao & | | | |
| Udagawa | | | |
| Myceliophthora | Sporotrichum | 45-50 | 55 |
| thermophila (Apinis) | thermophilum/thermophile, | | |
| van Oorschot | Chrysosporium | | |
| | thermophilum, | | |
| | Myceliophthora indica, | | |
| | Corynascus heterothallicus | | |
| Myriococcum | | 45 | 53 |
| thermophilum (Fergus) | | | |
| van der Aa | | | |
| Paecilomyces varioti | | 50 | 55 |
| Bainier^{b} | | | |
| Rhizomucor miehei | Mucor miehei | 35-45 | 57 |
| (Cooney & Emerson) | | | |
| Schipper | | | |
| Rhizomucor pusillus | Mucor pusillus | 35-45 | 55 |
| (Lindt) Schipper | | | |
| Scytalidium | Torula thermophila, | 40 | 58 |
| thermophilum (Cooney | Humicola grisea var. | | |
| & Emerson) Austwick | thermoidea, Humicola | | |
| | insolens | | |
| Stilbella thermophila | | 35-50 | 55 |
| Fergus | | | |
| Talaromyces | Paecilomyces | 40-45 | >50 |
| byssochlamydioides | byssochlamydioides | | |
| Stolk & Samson | | | |
| Talaromyces emersonii | Geosmithia emersonii; | 40-45 | 55 |
| | Talaromyces duponti and | | |
| | Penicillium duponti | | |
| | (misapplied names) | | |
| Talaromyces | Penicillium duponti | 45-50 | 60 |
| thermophilus | | | |
| Thermoascus | Thermoascus aurantiacus | 49-52 | 61 |
| aurantiacus | sensu Cooney & Emerson | | |
| | (misapplied name) | | |
| Thermomyces | | 42-47 | 61 |
| ibadanensis Apinis & | | | |
| Eggins | | | |
| Thermomyces | Humicola lanuginosa | 45-50 | 60 |
| lanuginosus | | | |
| Tsiklinskaya | | | |
| Thermomyces stellatus | Humicola stellata | 40 | 50 |
| (Bunce) Apinis | | | |
| Thielavia australiensis | | 35-40 | 50 |
| Tansey & Jack | | | |
| Thielavia pingtungia | | 40 | >50 |
| Chen K.-Y. & Chen | | | |
| Z.-C. | | | |
| Thielavia terrestris | Allescheria terrestris, | 40-45 | 52 |
| (Apinis) Malloch & | Acremonium alabamensis | | |
| Cain | | | |

^{a}Temperature data are from various sources and should be regarded as approximate. Because of uncertainty about the minimal temperature of growth (see text), this is not given.
T_{Opt}, optimal temperature;
Tₘₐₓ, maximum temperature.
^{b}Confusion exists regarding its designation as a thermophilic fungus.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and/or patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A composition of matter comprising:
(a) a nitrogen containing compound, an explosive or a substance used in the preparation of an explosive; and
(b) a spore of a micro-organism.

2. The composition of claim 1, wherein said micro-organism cannot degrade an explosive.

3. The composition of claim 1, wherein said micro-organism cannot degrade 2,4,6-trinitrotoluene.

4. The composition of claim 1, wherein said micro-organism is a bacteria.

5. The composition of claim 1, wherein said micro-organism is an extremophile.

6. The composition of claim 1, wherein said micro-organism is an auxotroph.

7. The composition of claim 1, wherein said micro-organism has a resistivity to an antibiotic.

8. The composition of claim 1, wherein said micro-organism includes a DNA barcode.

9. The composition of claim 1, wherein said composition is a solid.

10. The composition of claim 1, wherein said nitrogen containing compound is a fertilizer.

11. The composition of claim 1, wherein said explosive is selected from the group consisting of NT 2-nitrotoluene; NT 3-nitrotoluene; NT 4-nitrotoluene; TNT 2,4,6-trinitrotoluene; DNT 2,4-dinitrotoluene; DNT 3,4-dinitrotoluene; DNT 2,6-dinitrotoluene; EGDN ethylene glycol dinitrate; NG nitroglycerine; RDX cyclotrimethylenetrinitramine (cyclonite); PETN pentaerythritol tetranitrate; HMX homocyclonite (octogen); NH₄NO₃ Ammonium nitrate; NitroBid 1,2,3-propanetrial trinitrate Formulations; C-4 RDX and/or PETN Semtex RDX and/or PETN Detasheet RDX and/or PETN Dynamites EDGN and/or NG.

12. A method of determining a source of a material the material being an explosive or a substance used in the preparation of the explosive, the material comprises a spore of a micro-organism uniquely associated with the material, the method comprising inducing the spore to transform into a vegetative micro-organism and identifying the micro-organism as being associated with the material, thereby determining the source of the material.

13. The method of claim 12, wherein said micro-organism cannot degrade an explosive.

14. The method of claim 12, wherein said micro-organism cannot degrade 2,4,6-trinitrotoluene.

15. The method of claim 12, wherein said micro-organism is a bacteria.

16. The method of claim 12, wherein said micro-organism is an extremophile.

17. The method of claim 12, wherein said micro-organism is an auxotroph.

18. The method of claim 12, wherein said micro-organism has a resistivity to an antibiotic.

19. The method of claim 12, wherein said micro-organism includes a DNA barcode.

20. The method of claim 12, wherein said explosive is selected from the group consisting of NT 2-nitrotoluene; NT 3-nitrotoluene; NT 4-nitrotoluene; TNT 2,4,6-trinitrotoluene; DNT 2,4-dinitrotoluene; DNT 3,4-dinitrotoluene; DNT 2,6-dinitrotoluene; EGDN ethylene glycol dinitrate; NG nitroglycerine; RDX cyclotrimethylenetrinitramine (cyclonite); PETN pentaerythritol tetranitrate; HMX homocyclonite (octogen); NH₄NO₃ Ammonium nitrate; NitroBid 1,2,3-propanetrial trinitrate Formulations; C-4 RDX and/or PETN Semtex RDX and/or PETN Detasheet RDX and/or PETN Dynamites EDGN and/or NG.

21. A composition of matter comprising:
(a) a substance used in the preparation of a narcotic or a narcotic; and
(b) a spore of a micro-organism.
